(19) United States
(12) Patent Application Publication
(10) Pub. No.: US 2006/0263826 A1
(43) Pub. Date: Nov. 23, 2006

(54) Antibody protein analysis chip

(75) Inventor: Patrick J. Muraca, Pittsfield, MA (US)

Correspondence Address: SONNENSCHEIN NATH & ROSENTHAL LLP;FOR PAULA EVANS, P.O. BOX 061080, WACKER DRIVE STATION, SEARS TOWER, CHICAGO, IL, 60606-1080, US

(73) Assignee: Nuclea Biomarkers LLC

(21) Appl. No.: 11/436,246

(22) Filed: May 18, 2006

Related U.S. Application Data
(60) Provisional application No. 60/682,166, filed on May 18, 2005.

(51) Int. Cl. G01N 33/53; C12M 1/34

(52) U.S. Cl. 435/7.1; 435/287.2; 977/900

(57) ABSTRACT

The various embodiment described herein disclose an antibody protein analysis chip. The analysis chip comprises a substrate having a plurality of layers. At least one layer is a conductive layer comprising a conductive material. The analysis chip also comprises a main channel (straight-line and S-shaped configurations) etched in the conductive layer wherein the channel begins at a loading reservoir and ends at an outlet channel. A plurality of side channels are etched in the conductive layer. The side channels are in communication with the main channel and end in a well. A plurality of wells of the analysis chip comprise at least one protein-specific antibody. In addition, a kit is disclosed comprising an embodiment of the antibody protein analysis chip.

### RELATED APPLICATIONS

This application claims priority under 35 U.S.C. §119(e) to U.S. Provisional Application Ser. No. 60/682,166, filed May 18, 2005, the entirety of which is incorporated herein by reference.

### GOVERNMENT SUPPORT

None.

### FIELD OF THE INVENTION

The invention relates to the field of protein analysis; more specifically, to an antibody protein analysis chip for the separation, detection and identification of proteins.

### BACKGROUND OF THE INVENTION

In fields such as proteomics, large amounts of information must be gathered and analyzed as rapidly, accurately and efficiently as possible. Protein analysis chip technology has proven to be a valuable tool for performing such analysis.

A time limiting and costly step in such analysis is chip preparation and fluid dynamics. Analysis chips need to be carefully and accurately etched to produce a desired configuration. In addition, intricate electrode assemblies must be utilized in order provide a force (i.e., a charge) to accurately deliver a sample across the chip to a desired location. Each of these elements is costly and time consuming.

As such, there is a need in the art for an improved analysis chip for protein separation, detection and identification that is cost effective and does not require intricate electrode assemblies to transport a sample. In addition, these is a need in the art for a protein analysis chip wherein samples may be easily and accurately transported across a analysis chip to a desired location on the chip.

### SUMMARY OF THE INVENTION

The various embodiment described herein disclose an antibody protein analysis chip. In a presently disclosed embodiment, the analysis chip comprises a substrate having a plurality of layers. At least one layer is a conductive layer comprising a conductive material. The analysis chip also comprises a main channel etched in the conductive layer which begins at a loading reservoir and ends at an outlet channel. A plurality of side channels are etched in the conductive layer at a substantially perpendicular angle to the main channel wherein the side channels are in communication with the main channel and end in at least one well. A plurality of wells of the analysis chip comprise at least one protein-specific antibody.

In an embodiment, the use of a conductive material in combination with the orientation of the main channel and side channels allows for an alternating current to be established along the main channel; this embodiment allows for an inexpensive design while being capable of accurate sample transport.

In a presently disclosed embodiment, the analysis chip comprises an S-shaped channel etched in the conductive layer which begins at a loading reservoir and ends at an outlet channel. A plurality of side channels are etched in the conductive layer wherein the side channels are in communication with the main channel and end in a well. A plurality of wells of the analysis chip comprise at least one protein-specific antibody confined in each well. In addition, a energy source (i.e., a battery) is in electrical communication with the substrate to allow for a current to be applied to the conductive layer. In an embodiment, an alternating current is established along the chip to provide for accurate sample transport. In addition, the S-shaped channel allows for enhanced protein separation.

In another embodiment, a kit is disclosed wherein the kit comprises an embodiment of the antibody analysis chip engaged to a cover. The cover engages to the analysis chip and comprises a plurality of openings corresponding to the wells. In an embodiment, the kit comprises protein-specific antibodies confined to the various wells.

In other embodiments, various methods of using the antibody protein analysis chip for protein separation, identification and analysis are disclosed.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows an embodiment of a antibody protein analysis chip wherein a main channel comprises a straight-line configuration;

FIG. 2 shows an embodiment of the antibody protein analysis chip comprising a plurality of main channels having a straight-line configuration;

FIG. 3 shows an embodiment of the antibody protein analysis chip wherein a main channel engages a plurality of wells along the main channel;

FIG. 4 shows an embodiment of the antibody protein analysis chip wherein a main channel comprises an S-shaped configuration;

FIG. 5 shows an embodiment of a antibody protein analysis chip comprising a plurality of main channels each having an S-shaped configuration;

FIG. 6 shows an embodiment of the antibody protein analysis chip comprising an S-shaped configuration wherein a plurality of wells are positioned in the main channel;

FIG. 7A shows a side view of an embodiment of a substrate wherein the substrate comprise three layers. FIG. 7B shows a side view of an embodiment of a substrate wherein the substrate comprises four layers; and

FIG. 8 shows a one-dimensional gel of serum protein following separation on the antibody protein analysis chip.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The various embodiments disclosed herein relate to an antibody protein analysis chip for use in protein separation, identification and analysis. The embodiments disclose a chip comprising a conductive material which facilitates the delivery and control of samples (i.e., a protein mixture) across the analysis chip. Sample delivery is facilitated because the conductive material allows for a charge to be maintained across the chip. Maintaining and controlling the charge across the chip allows for the efficient and accurate delivery of sample to desired locations across the chip. In addition, the use of a conductive material eliminates the need for intricate and complicated electrode assemblies for the movement of a sample. As a further advantage, conductive materials are easier and less expensive to etch than glass chips. Therefore, channels, side channels and wells may be easily fabricated into the chip.

Further embodiments disclosed herein provide an “S-shaped” main channel. The S-shaped channel allows for enhanced protein separation. As the sample proceeds from a loading channel (entrance) to an output channel, the various curves of the S-shaped main channel allow for separation of the various constituents of the sample.

Various embodiments herein disclose an analysis chip comprising a substrate having a conductive layer. Channels are etched in the conductive material so as to allow a sample to pass across the chip. A main channel (running from the loading reservoir to the outlet channel) engages various side channels. In various embodiments disclosed herein the side channels engage a well containing a protein-specific antibody.

The use of the conductive material allows for a charge to be maintained across the analysis chip thereby facilitating the ability to accurately deliver a sample across the chip and into a desired channel or well. In various embodiments, the alignment of the main channel and various side channels in combination with the characteristics of the conductive material creates an alternating current across the analysis chip. In other embodiments, an energy source (i.e., a battery) is used to provide the alternating current across the chip.

In the various embodiments, the antibody protein analysis chip comprises protein-specific antibodies located at various wells across the chip. The features of the chip allow for a sample to be accurately directed to each well and come into contact with the various antibodies. In an embodiment, the antibodies selectively bind a desired protein (if the protein is present in the sample). In an embodiment, the antibodies are specific for an undesired protein and allow the desired protein to leave the chip through the outlet channel.

In an embodiment, the antibodies are engaged to a detectable tag. Detectable tags are used to determine when/if a protein has become bound by any of the various antibodies.

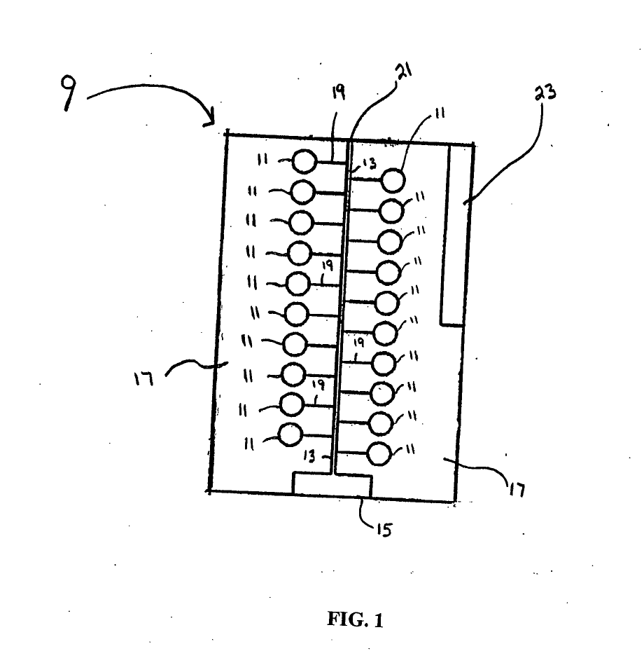

FIG. 1 shows an embodiment of an antibody protein analysis chip 9 comprising a substrate 17. A cover slip (not shown) may be engaged to the substrate 17 in order to provide a closed environment.

In an embodiment, the substrate 17 comprises a plurality of layers. In an embodiment, a first layer comprises glass. The glass is engaged to a second layer. In an embodiment, the second layer comprises chrome. Those skilled in the art will recognize that the second layer may comprise a variety of materials and remain within the spirit and scope of the present invention.

In an embodiment, the first layer is engaged to the second layer by electroplating the second layer to the first layer. Those skilled in the art will recognize that various techniques may be used to engage the first layer to the second layer and remain with the spirit and scope of the present invention.

In an embodiment, the second layer may be engaged to a third layer. In an embodiment, the third layer is a conductive layer comprising a conductive material. In an embodiment, the third layer comprises gold. (Chips comprising gold are commercially available at Erie Scientific, Portsmouth, N.H.). In an embodiment, the third layer comprises aluminum. In an embodiment, the third layer comprises chromium. Those skilled in the art will recognize that the third layer may comprise a variety of conductive materials and remain within the spirit and scope of the present invention.

In an embodiment, the second layer is engaged to the third layer by electroplating the third layer to the second layer. Those skilled in the art will recognize that various techniques may be used to engage the third layer to the second layer and remain with the spirit and scope of the present invention.

Various channels and wells are etched into the substrate 17 by use of a laser. Those skilled in the art will recognize that various technologies may be used to create the desired channels and/or wells in the substrate 17 and remain within the spirit and scope of the present invention.

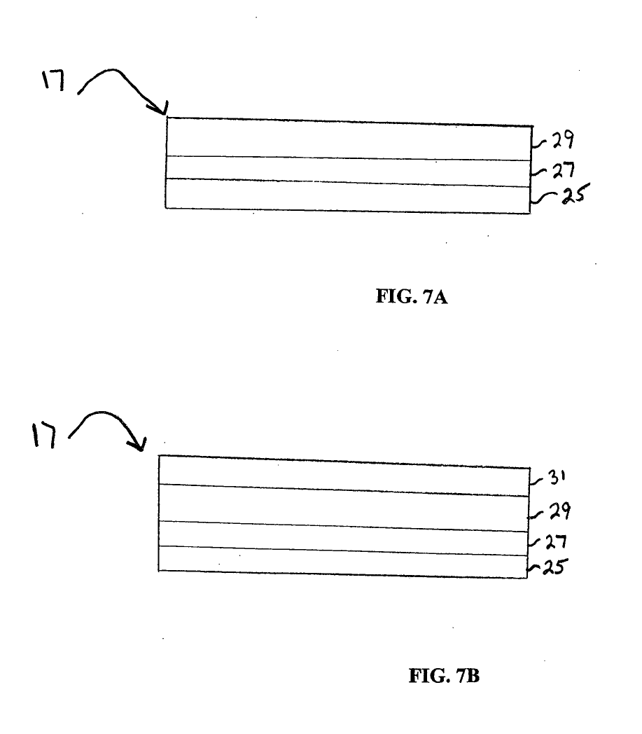

The channels and wells of the analysis chip are etched into the third layer of the substrate 17. In an embodiment, the channels and wells of the analysis chip are etched into the third layer and the second layer of the substrate 17. In an embodiment, the channels and/or wells of the analysis chip are etched into a gold third layer of the substrate 17 and a chrome second layer of the substrate 17 wherein such materials are easier and cheaper to etch than glass. FIGS. 7A and 7B (discussed below) illustrate a side view of various embodiments of a substrate.

As shown in FIG. 1, the substrate 17 comprises a loading reservoir 15 for the introduction of a sample. The sample may comprise a plurality of proteins (i.e., a plurality of low abundant proteins.) In an embodiment, it is unknown whether or not the sample comprises a desired protein (in the case of protein detection). In an embodiment, the sample comprises a desired protein mixed with a plurality of undesired proteins (in the case of protein separation). Those skilled in the art will recognize that various samples are within the spirit and scope of the present invention.

In an embodiment, the loading reservoir 15 is engaged to a main channel 13 at a first end and engages an outlet channel 21 at a second end. A plurality of side channels 19 are engaged to the main channel 13. In an embodiment, the plurality of side channels 19 are substantially perpendicular to the main channel 13. In an embodiment, the plurality of side channels 19 are not perpendicular to the main channel 13. Those skilled in the art will recognize that the plurality of side channels 19 may engage the main channel 13 at various angles and remain within the spirit and scope of the present invention.

At least one side channel 19 is engaged to an at least one well 11. In an embodiment, a side channel 19 engages a plurality of wells 11. In an embodiment, a plurality of the side channels 19 are engaged to the well 11. In an embodiment, a diameter of a well 11 is about 3 mm. Those skilled in the art will recognize that the wells may be of a wide range of diameters and remain within the spirit and scope of the present invention.

In an embodiment, each well comprises an antibody. In an embodiment, each antibody is capable of specifically binding to a protein. In an embodiment, at least one antibody is a kinase related antibody. Provisional Application No. 60/682,115, (entitled Kinase Peptides and Antibodies; filed May 18, 2005) and co-pending U.S. application No. ______, (entitled Kinase Peptides and Antibodies; filed May 17, 2006) the entirety of which are incorporated herein by reference, disclose various antibodies which may be used in conjunction with the present invention.

In an embodiment, an protein-specific antibody is engaged to a detectable tag. The detectable tag allows for a determination of when/if a protein has been bound to the tagged antibody. In an embodiment, the detectable tag is a quantum dot. In an embodiment, the detectable tag is a fluorescent dye. Those skilled in the art will recognize that various detectable tags are within the spirit and scope of the present invention.

A quantum dot is a semiconductor crystal with a diameter of a few nanometers, also called a nanocrystal, that because of its small size behaves like a potential well that confines electrons in three dimensions to a region on the order of the electrons' de Broglie wavelength in size, a few nanometers in a semiconductor. Because of the confinement, electrons in the quantum dot have quantized, discrete energy levels, much like an atom. For this reason, quantum dots are sometimes called “artificial atoms”. The energy levels can be controlled by changing the size and shape of the quantum dot, and the depth of the potential.

Many scientific applications require materials that absorb and fluoresce at specific wavelengths. However, standard materials often have limited absorptive capabilities because their absorption spectrum is either too narrow or their absorption onsets cannot be easily changed. For example, bulk semiconductor crystals have a broad absorption spectrum, but it is very flat and therefore difficult to manipulate. Organic dyes have distinct absorption peaks, but they tend to be narrow, non bell-shaped, and not easily tuned. Quantum dots combine the broad absorption spectrum of a semiconductor crystal with the distinct absorption peak of an organic dye, in addition to having longer fluorescent lifetimes. Finally, quantum dots can be tuned to emit in sharp, Gaussian peaks at any visible or infrared frequency.

In an embodiment, each antibody is engaged to their respective well 11 so that the antibody is substantially immobilized within the well 11. In an embodiment, an antibody or an antibody-detectable tag complex is engaged to a well 111 by a poly-L-lysine. In an embodiment, an antibody or an antibody-detectable tag complex is engaged to a well 11 through a silanization technique. The silanization technique comprises heating the glass slide to above about 100° C. and de-hydrating the glass. Next, the glass may then be exposed to a silane (under a vacuum to increase the efficiency of the process.) The silane may then take a desired configuration prior to hydrolyzing the glass with water. To better understand the process, one may think of silane as having three leaving groups wherein the leaving groups will either crosslink to itself or may crosslink to the glass (or any oxide surface). The other end (which is spaced a number of carbons away) may comprise any desired functional group. The functional group is any group that may bind an antibody. In an embodiment, the functional group is an amine group. In an embodiment, the functional group is an aldehyde group. In an embodiment, the functional group is an epoxy group. In an embodiment, the functional group is a mercapto group. In an embodiment, the functional group is a hydroxyl group. In an embodiment, a C═C group is engaged to the silane. Next, the C═C is hydroborated and oxidized to produce a desired functional group. Those skilled in the art will recognize that various functional groups are within the spirit and scope of the present invention.

In an embodiment, a bar code 23 is engaged to the substrate 17 and may be used as an identifier for a particular analysis chip 9. The bar code 23 may be scanned by a scanner (not shown) wherein the scanner is engaged to a computer (not shown). As such, the identify of the analysis chip 9 may be stored in a computer via the bar code 23. Various types of information may be associated with the bar code of an analysis chip 9. As examples, information such as the identity of a sample, sample size, time and day of experiment, experimental conditions, and information regarding the sample source may be associated with the bar code in a database of the computer. In addition, various types of information elucidated from the method of the invention (such as results from a mass spectrometer, signal generated by a detectable tag, etc) may be inputted into the database and correlated to the appropriate analysis chip via the bar code 23. As such, the use of bar codes 23 facilitate the use of multiple chips at once thereby enhancing efficiency and productivity.

The outlet channel 21 may be engaged to an additional downstream separation and or detection device or an additional separation device. In an embodiment, the outlet channel 21 is engaged to a mass spectrometer. Those skilled in the art will recognize that outlet channel may be engaged to various downstream devices and/or instrumentation and remain within the spirit and scope of the present invention.

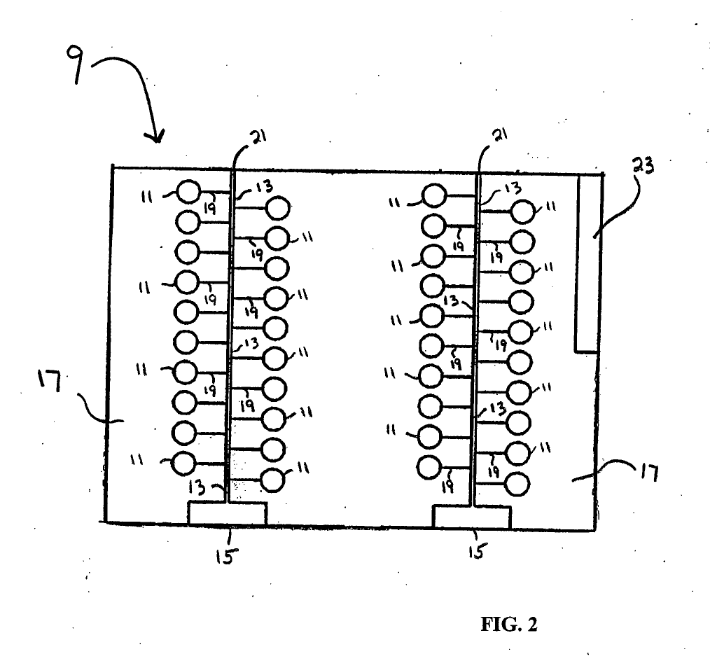

FIG. 2 shows an embodiment wherein the substrate 17 comprises a first main channel 13 engaging a plurality of side channels 19 and a second main channel 13 engaging a second plurality of side channels 19. In an embodiment, the substrate 17 comprises a third main channel (not shown) engaged to a third plurality of side channels. Those skilled in the art will recognize that a substrate 17 comprising any number of main channels (along with any number of side channels) may be within the spirit and scope of the present invention.

FIG. 1 and FIG. 2 show embodiments of the invention wherein the main channel 13 comprises a straight line configuration. The main channels 13 of FIG. 1 and FIG. 2 engage a plurality of side channels 19 at substantially right angles. As will be described below, the use of a substrate having a conductive layer in combination with the use of a straight main channel 13 engaging side channels 19 at substantially perpendicular angles allows for an alternating charge to be maintained across the surface of the analysis chip. As such, etched channels of a specific orientation in a conductive material allow for a simplified electronic circuit.

In an embodiment, a charge propagates across the antibody protein analysis chip 9 (from the loading reservoir 15 to the outlet channel 21). The charge propagates because the substrate 17 of the analysis chip 9 comprises a conductive material. As the charge engages each successive side channel 19, the charge alternates from a positive (+) charge to a negative (−) charge. As such, the use of a conductive material in combination with a substantially straight line configured main channel 13 engaging a plurality of substantially perpendicular side channels 19 results in an alternating current along the surface of the analysis chip 9. The alternating current facilitates the accurate delivery of the sample through the main channel 13, through the side channels 19, and into a desired well 11. As such, the various embodiments disclosed herein comprise a low cost device with simplified design characteristics (minimal features needed to maintain an alternating current) of analysis chip 9.

As another advantage of the disclosed chip design, the alternating current facilitates delivery of a sample to a well 11 and allows for better contact (and assists in delivering a substantial amount of sample to each well 11) between the protein-specific antibody in the well 11 and the sample. Longer retention times and larger amounts of sample being delivered to each well allows for improved and more accurate results.

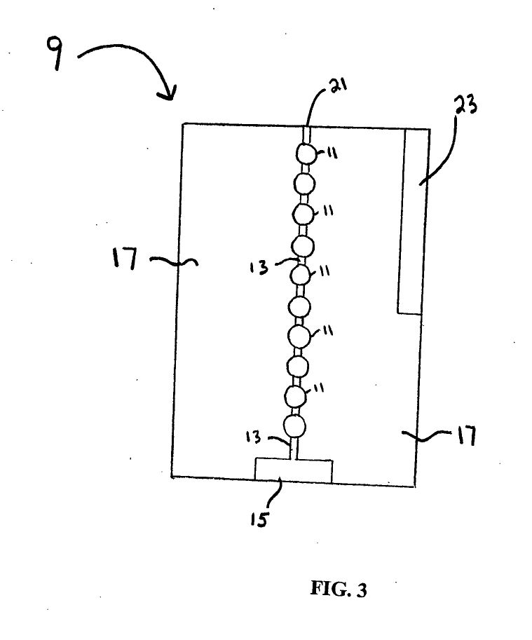

FIG. 3 shows an embodiment wherein an analysis chip comprises a main channel 13 having a straight-line configuration wherein a plurality of wells 11 are located in the main channel 13. Placing the wells 11 in the main channel facilitates the delivery of the sample to the well 11 (a substantial amount of the sample comes into contact with each well and therefore with each protein-specific antibody) and thereby maximizes antibody contact with the sample. In an embodiment, the main channel 13 comprises a plurality of wells 111 in the main channel 13 (as shown in FIG. 3) in addition to engaging the main channel 13 to a plurality of side channels 19 wherein each side channel 19 engages a well 11.

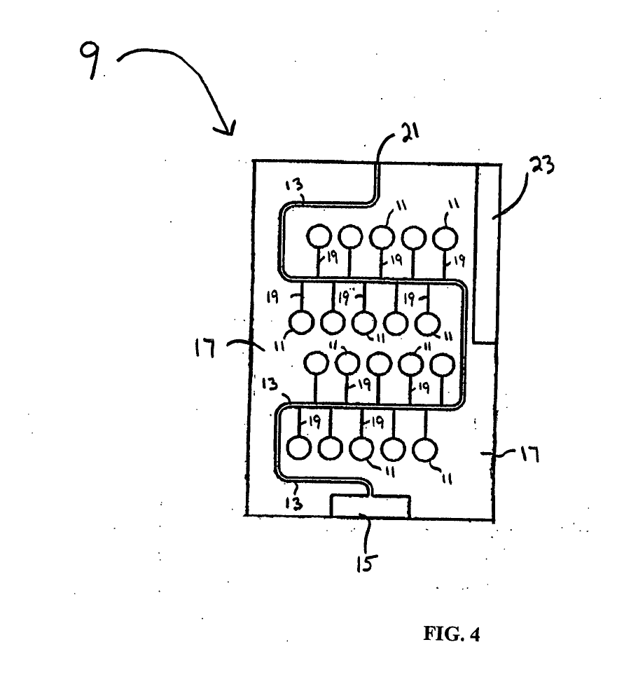

FIG. 4 shows an embodiment of the analysis chip 9 comprising a main channel 13 of an “S-shaped” configuration. A sample is loaded into a loading reservoir 15 and a cover slip (not shown) is engaged to the analysis chip 9. The presence of the cover slip creates an embodiment wherein a portion of a sample in the loading reservoir 15 is drawn into the main channel 13 by a capillary action. In an embodiment, the capillary action helps draws the sample through main channel 13 of the analysis chip 9 and into the plurality of side channels 19.

A main channel 13 comprising an S-shaped configuration provides various unexpected benefits. The S-shaped configuration provides an improved capillary force for driving a sample through an analysis chip 9. In addition, the S-shaped configuration facilitates the separation of proteins in a sample. As such, the distinct proteins will separate from each other as the sample encounters the various turns of the S-shaped main channel 13. As a further benefit, increased protein separation along the curves of the S-shaped configuration results in an improved binding efficiency between a desired protein and an antibody in a well 11.

In an embodiment, an alternating current is generated across an analysis chip 9 having an S-shaped configuration by an energy source. In an embodiment, the energy source is a battery. Those skilled in the art will recognize that an alternating current generated by any of several techniques is within the spirit and scope of the present invention. As discussed above, the conductive layer of the chip 9 facilitates the use of the alternating current; the alternating current provides a very accurate means of delivering a sample to a desired location of the chip 9.

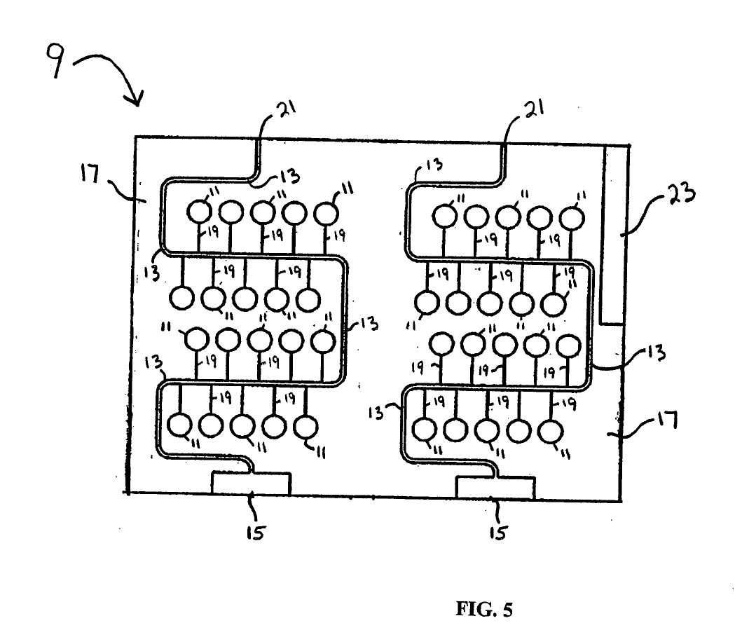

FIG. 5 shows an embodiment wherein the substrate 17 comprises a first main channel 13 having an S-shaped configuration engaging a plurality of side channels 19 and a second main channel 13 having an S-shaped configuration engaging a second plurality of side channels 19. In an embodiment, the substrate 17 comprises a third main channel (not shown) having an S-shaped configuration engaged to a third plurality of side channels (not shown). Those skilled in the art will recognize that a substrate 17 comprising any number of main channels (along with side channels) may be within the spirit and scope of the present invention.

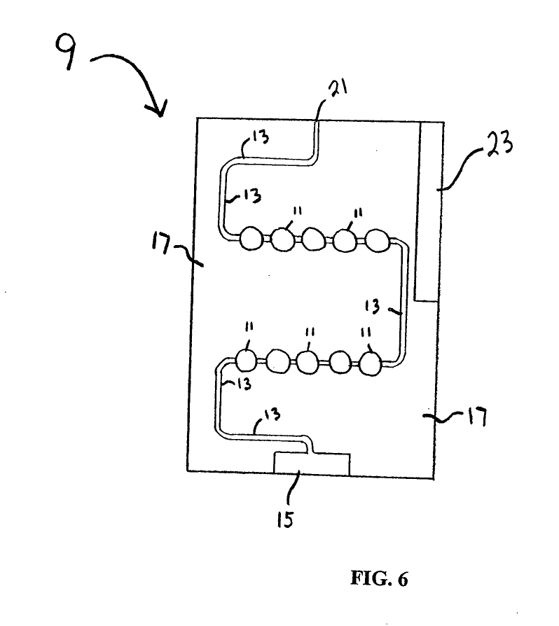

FIG. 6 shows an embodiment wherein an analysis chip comprises a main channel 13 having an S-shaped configuration. In an embodiment, a plurality of wells are located in the main channel 13. Placing the wells 11 in the main channel 13 facilitates the delivery of the sample to the well and thereby maximizes antibody contact with the sample. In an embodiment, the main channel 13 comprises a plurality of wells 11 in the main channel 13 (as shown in FIG. 6) in addition to engaging a plurality of side channels 19 wherein each side channel engages a well 11.

FIG. 7A shows a side view of an embodiment of a substrate of an analysis chip. In an embodiment, the substrate 17 comprises a plurality of layers. In an embodiment, the substrate 17 comprises a glass layer (a glass slide) 25. In an embodiment, the substrate 17 comprises a second layer 27 engaged to the glass layer 25. The second layer 27 may be engaged to the glass layer 25 by electroplating. In an embodiment, the second layer 27 comprises chrome. In an embodiment, the substrate 17 comprises a third layer 29 engaged to the second layer 27. In an embodiment, the third layer 29 is electroplated to the second layer 27. In an embodiment, the third layer 29 comprises a conductive material. In an embodiment, the third layer 29 (the conductive layer) comprises gold. In an embodiment, the third layer 29 comprises aluminum. In an embodiment, the third layer 29 comprises chromium. Those skilled in the art will recognize that a variety of conductive materials are within the spirit and scope of the present invention.

A plurality of channels and/or wells are etched in the third layer 29 and/or second layer 27. An advantage of these various embodiment is that the materials which comprise the second layer 27 and the third layer 29 are easier to etch than a glass microchip; this advantage leads to much lower costs.

FIG. 7B shows an embodiment of the invention wherein a fourth layer 31 is engaged to the third layer 29. The fourth layer assists in retaining a sample in a channel. In an embodiment, the fourth layer 31 comprises a hydrophobic material. In an embodiment, the fourth layer 31 comprises an ink. In an embodiment, the fourth layer 31 comprises a hydrophobic layered ink. The hydrophobic nature of the fourth layer 31 repels a sample and as such retains the sample in a desired channel of the analysis chip 9.

Various embodiments are disclosed for protein identification, separation and analysis.

Method of Using the Device to Separate a Protein Mixture

In an embodiment, a desired protein may be separated from a mixture of proteins and/or other materials in order to perform additional analysis on the desired protein. The mixture, comprising the desired protein and a plurality of unwanted proteins and/or other materials, is delivered to a loading reservoir 15 of an analysis chip 9. The mixture is drawn into the main channel 13 of the analysis chip 9 by a capillary action and propagated down the main channel 13 by an alternating charge present on the analysis chip 9. As the mixture travels down the main channel, application of a force (i.e., the charge) directs the sample into a desired well 11. A protein-specific antibody is located in each well 11. In an embodiment, each antibody is specific for an undesired protein or peptide of the mixture. As such, as the mixture comes into contact with an antibody in a well 11, the antibody will remove the undesired protein from the mixture. The mixture then travels out of the side channel 19 and into the main channel 13 of the analysis chip 9. The mixture next travels to a second reaction well 11 which comprises a second protein specific antibody. In an embodiment, the antibody in the second reaction well binds to an additional undesired protein of the sample. The sample then travels out of the second well 11 and into the main channel 13 for further processing. In an embodiment, the process continues down the length of the main channel 13 of the analysis chip 9 until the sample substantially comprises only the desired protein (a protein which does not bind to the various antibodies that the sample encounters in the various wells 11) and the majority of the undesired protein has been eliminated from the sample by binding to various antibodies in the various wells 11 and thereby being confined to the various wells 11. As such, the desired protein travels out of the outlet channel 21 of the analysis chip 9. The outlet channel 21 may engage various other instrumentation in order to further analysis or process the desired protein.

In an embodiment, each well 11 comprises the same antibody. In an embodiment, each well 11 comprises a different antibody. In an embodiment, the number and type of antibody is based upon the number and type of undesired proteins in the sample (i.e., there should be sufficient type and quantity of antibody to bind substantially all of the undesired protein). In an embodiment, at least one well 11 comprises an antibody which is different from an antibody in a second well 11. In an embodiment, a single well may comprise multiple antibodies.

In an embodiment, the antibodies are specific for the desired proteins. As such, as the mixture travels through the analysis chip and into each well, the antibodies bind to the desired proteins and the remaining (unwanted) material is allowed to exit the chip by the outlet channel.

Method of Using the Device for Protein Identification

In an embodiment, the analysis chip 9 may be used to identify the presence (or absence) of a desired protein. A sample is provided wherein it is unknown whether or not a desired protein or a plurality of proteins are in the sample. The method of this embodiment may be utilized to identify various biomarkers or used in a variety of other diagnostic tests.

A sample is placed in a loading reservoir 15 of the analysis chip 9. At the outset, it is unknown whether or not the sample comprises an individual or a plurality of proteins of interest (i.e., the desired proteins). As the sample travels down the main channel 13 to a desired well 11, the sample is directed into the well 11 and engages a protein-specific antibody-detectable tag complex.

The antibody of the antibody-detectable tag complex is capable of binding a specific protein. More specifically, the antibody specifically binds to the desired protein (whose presence is an unknown in the sample.) In other words, a sample may or may not comprise Protein A. In this case, the antibody of the antibody-detectable tag complex specifically binds to Protein A (if Protein A is present in the sample).

In an embodiment, the sample travels to a well 11 and comes into communication with the first antibody-quantum dot complex wherein the antibody is specific for the desired protein. If the sample contains the desired protein, the protein will bind to the antibody due to the fact that the antibody has been selected to specifically bind to the protein. Obviously, if the desired protein is not present in the sample then the antibody will remain unbound (as will be indicated by the detectable tag).

In an embodiment, the quantum dot of the antibody-detectable tag complex is capable of generating a signal if the antibody of the antibody-detectable tag complex binds to the desired protein. In an embodiment, the detectable tag does not generate a signal if the antibody does not bind to the desired protein. In an embodiment, the signal generated by a detectable tag may be detected and measured.

In an embodiment, a sample may be tested for a plurality of desired proteins. In such a case, a plurality of wells 11 each comprises antibodies which selectively bind different proteins. In an embodiment, each antibody specifically binds to a different desired protein. Each of the antibodies is engaged to a detectable tag (i.e., a quantum dot). A sample is delivered to a plurality of wells so that a plurality of distinct antibodies may come into communication with the sample. The desired proteins of a sample may engage a plurality of different antibodies on a single analysis chip 9. Each of the distinct antibody-detectable tag complexes generate a different signal indicating that the various antibody-detectable tag complexes are each bound to a distinct desired protein. In an embodiment, the analysis chip 9 may identify a plurality of kinase related antibodies (as identified in U.S. Provisional Application No. 60/682,115, (entitled Kinase Peptides and Antibodies; filed May 18, 2005) and co-pending U.S. application Ser. No. ______, filed on May 17, 2006, the entirety of which are incorporated herein by reference).

Production Kit

The analysis chip may comprise a production kit. In an embodiment, an analysis chip 9 will be placed in a holder (not shown). The holder may comprise a plastic and/or polymer material. A cover will be placed on top of the analysis array 9. The cover will comprise a plurality of holes wherein the holes correspond to the various wells 11. A plurality of antibody-detectable tag complexes may be added to each well through the various holes. As such, a kit is herein disclosed wherein the analysis chip may be sold in a contained environment while a plurality of desired antibodies are added to the various wells 11.

Consumer Kit

The presently disclosed embodiments may comprise a kit having an analysis chip with a plurality of antibodies deposited in a plurality of wells wherein a cover is placed over the chip 9. The cover may comprise a plastic and/or polymer material. The cover may comprise an opening over the loading reservoir thereby enabling the introduction of sample while maintaining a clean environment on the remainder of the analysis chip. Customers may request a kit having specific antibodies in the various wells; as such, a customer may order a kit specific to their needs.

### EXAMPLE

The following example is merely illustrative of various features of an embodiment of the antibody protein analysis chip. The example is not meant to limit any aspect of any embodiment of the analysis chip.

As an overview, the following example illustrates the following: first, albumin is removed from a serum sample; second, one-dimensional gel electrophoresis is utilized in order to confirm removal of albumin; third, the resulting serum sample (which is now albumin free) is loaded into an embodiment of the antibody protein analysis chip. The sample comprises various unwanted proteins along with proteins of interest. The chip comprises antibodies positioned in various wells (as described above) which specifically bind to unwanted proteins. As a result, unwanted proteins are removed from the sample as the unwanted proteins bind to the antibodies in the wells. Therefore, a sample comprising only (or substantially only) the proteins of interest exit the chip through the outlet channel.

Once the sample has exited the chip, a one-dimensional electrophoresis is run again in order to confirm the presence of the proteins of interest in the sample. The example is meant to illustrate the use of the chip in combination with albumin removal procedures for the separation of low abundance proteins from a serum sample. The various steps are described in detail below:

Study Objectives:

The purpose of the following study was:

1.) To screen serum samples from human colon (e.g. APC mutated and wild type) to identify protein markers that may be specific for colon polyps; and

2.) To develop a novel strategy for the solution-based isolation and identification of protein biomarkers from serum and/or plasma using chromatographic techniques and linked to an embodiment of the antibody protein analysis chip.

Specimens:

1.) Serum samples (about 0.05 mL aliquots) total of 22 tubes from three different mice. Three APC mutant mice and one wild type mouse.

One Dimensional Gel Electrophoresis and Albumin Depletion Procedures:

As a first step, albumin was removed from the sample by the following procedure:

1. Running ID SDS-PAGE gels using BioRad Criterion System

a. Load about 10 ul of the sample onto about 4-15% polyacrylamide SDS-PAGE gels.

b. Gels were placed into a BioRad Criterion system and run for about 1 hour 30 minutes at a constant voltage of about 200V.

c. Proceed with Coomassie staining (Bio-Rad).

2. Albumin Removal using Millipore Montage Albumin Depletion Kit.

a. Dilution of sample: about 100 μL of serum into about 100 μL of provided equilibration buffer
1. Column Equilibration
   a. Add about 400 μL of Equilibration Buffer to the column
   b. Spin at about 2000 rpm for about 2 minutes.
   c. Discard eluent
   d. Repeat 1×

b. Albumin Removal
1. Added diluted sample to the filter
2. Spin at about 2000 rpm for about 2 minutes
3. Recover the eluent and add back to the filter
4. Spin at about 2000 rpm for about 2 minutes
5. Recover the eluent and transfer to a provided collection tube
6. Label the tube “A−”

c. Washes
1. Add about 200 μL of wash buffer to the filter
2. Spin at about 2000 rpm for about 2 minutes.
3. Add another about 200 μL
4. Spin at about 2000 rpm for about 2 minutes.
5. Collect eluent and add to the A− collection tube

d. Recovering Albumin
1. Add about 400 μL of stripping buffer to the filter
2. Spin at about 2000 rpm for about 2 minutes.
3. Collect the eluent and place in an “A+” tube
   3. Acetone Precipitation

a. Aliquot about 200 μL of albumin depleted samples and add 3× of cold acetone (i.e., for about 200 μL add about 600 μL). Invert several times to mix.

b. Place in about 20° C. for about 2 hours.

c. Spin at about 12000 rpm for about 10 minutes.

d. Remove the supernatant and allow the pellets to air dry for approximately 10 minutes on ice.

Principles of the Procedures For Identifying Biomarkers in Serum:

Albumin Removal

Proteomic analysis of complex samples, such as plasma or serum is frequently influenced by the presence of high abundance proteins such as, albumin or immunoglobulin. The abundant serum proteins such as albumin hinder the analysis of low abundance proteins, therefore it is advantageous to specifically remove albumin prior to sample fractionation. Protocols for removing serum albumin from single samples have been developed. Montage Albumin Deplete kits (Catalogue # LSKAD004, Millipore Corp., Bedford, Mass.) were selected to process the serum samples. This product was selected based on trial experiments in which about 60-85% of the albumin was depleted with very low non-specific binding of other proteins. The albumin depleted serum allowed for higher resolution 2-D electrophoresis and solution based LC-MS to be performed.

Gel Electrophoresis

One-dimensional electrophoresis (1-D), SDS-polyacrylamide gel electrophoresis (SDS-PAGE), separates proteins according to their molecular weights (MW). Each spot on the resulting 1-D map corresponds to several protein species, therefore this technique is used to determine the quality of the samples prior to LC-MS/MS analysis. This technique provides a picture image of the samples.

Separation Using Antibody Protein Analysis Chip technology

Following the removal of albumin, the sample was added to the antibody protein analysis chip so that desired low abundance proteins may be separated from a mixture of proteins (mixture contains unwanted proteins and desired proteins). The sample was delivered to a loading reservoir of a analysis chip. The mixture comprises the desired protein and a plurality of proteins and/or other materials. The protein mixture was drawn into the main channel of the analysis chip and the charge present on the chip forced the protein into the chip wells. The protein traveled down the main channel and into various side channels of the chip. Each side channel was connected to a well. Specific Tyrosine Kinase antibodies were located in each well where the antibody was immobilized in the well via poly—L and each antibody is specific for an undesired protein or peptide of the mixture. As the protein mixture came into contact with the antibodies, the antibodies removed the undesired proteins from the mixture. The protein mixture then traveled out of the side channel and into the main channel of the microfluidic chip. The desired separated protein traveled out of the outlet channel of the microfluidic chip. The total measurement of the low molecular weight proteins were analyzed by fluorescent technology.

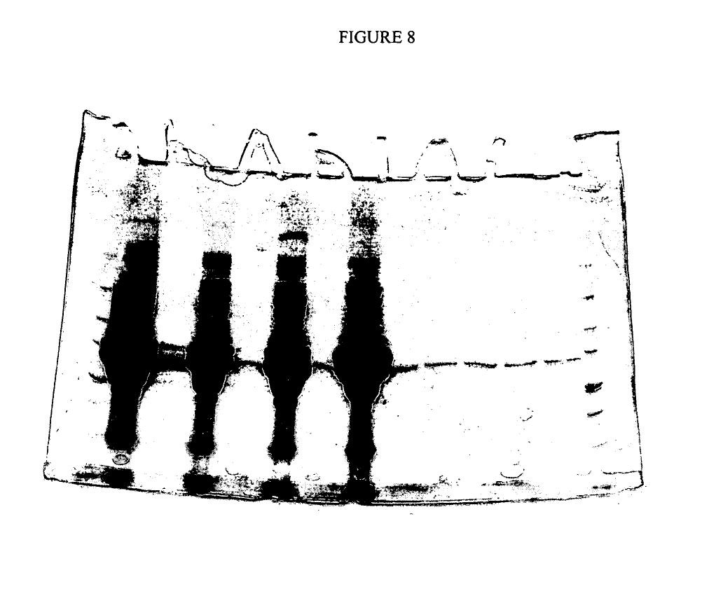

Next, a one-dimensional gel was run of the resulting sample produced by the antibody protein analysis chip. FIG. 8 shows the one-dimensional gel of serum protein following the above-describe separation. The protocol for the gel is identical to the one-dimensional gel protocol described above. In FIG. 8, the lanes are as follows: MW marker (Bio-Rad), APC-mutant colon 1, Blank, APC-mutant colon 2, Blank, APC-mutant colon 3, Blank, APC-wild type mouse, Blank, Blank, Blank, Blank, Blank, MW marker. The gel illustrates the successful removal of unwanted proteins from the mixture by use of the antibody protein analysis chip.

### Example 2

The following tables disclose antibodies which may be utilized with the antibody protein analysis chips. These tables are in no way meant to limit the number or type of antibodies that may be used with the chip. These tables are merely illustrative.

TABLE 1
Motif-specific antibodies successfully used (by Nuclea Biomarkers) for phosphoprotein
profiling in paraffin and frozen Tissue MicroArrays (‘+control’ binds the
unphosphorylated protein).

| | | | Kinase | | CST |
|---|---|---|---|---|---|
| | ST pathway | | substrate | | cat. |
| | profile | Phosphoprotein# | motif | Upstream kinase(s) | no.* |
| 1 | Akt/survival | AKT1 | Ser473 | PDK1/2 | 9277 |
| 2 | Akt/survival | AKT1 | +control | unphosphorylated | 9272 |
| 3 | Akt/survival | GSK-3α/β | Ser21/9 | AKT/PKB | 9331 |
| 4 | Akt/survival | FKHR/FKHRL1# | Thr24/Thr/32 | AKT/PKB | 9464 |
| 5 | Akt/survival | AFX | Ser193 | AKT/PKB | 9471 |
| 6 | Akt/survival | PTEN/MMAC | Ser380 | CK2 | 9551 |
| | | phosphatase | | | |
| 7 | Akt/survival | PTEN/MMAC | +control | unphosphorylated | 9556 |
| | | phosphatase | | | |
| 8 | Akt/survival | PDK1 | Ser241 | autophosphorylated | 3061 |
| 9 | Akt/survival | mTOR | Ser2448 | Akt | 2971 |
| 10 | PTK/survival | P70-S6 kinase | Thr421/Ser424 | PDK1/2 | 9204 |
| 11 | PTK/survival | S6 ribosomal protein | Ser240/244 | Erk, PKCζ, PDK1, | 2215 |
| | | | | FRAP/mTOR | |
| 12 | PTK/survival | S6 | +control | unphosphorylated | 2212 |
| 13 | RAS-MAPK | MEK1/2 | Ser217/221 | c-Raf | 9121 |
| 14 | RAS-MAPK | p44/42 Erk1/2 MAPK | Thr202/Tyr204 | MEK1/2 | 9101 |
| 15 | RAS-MAPK | p44/42 Erk1/2 MAPK | +control | unphosphorylated | 9102 |
| 16 | RAS-MAPK | P90RSK(1-3) | Thr359/Ser363 | Erk1/2 | 9344 |
| 17 | RAS-MAPK | P90RSK | +control | unphosphorylated | 9342 |
| 18 | RAS-MAPK | P38 MAPK | Thr180/Tyr182 | MKK3, MKK6 | 9211 |
| 19 | TK | Src family | Tyr416 | autophosphorylated | 2101 |
| 20 | TK | HER-2/ErbB2 | Tyr877 | autophosphorylated | 2241 |
| 21 | TK | HER-2/ErbB2 | +control | unphosphorylated | 2242 |
| 22 | TK | EGFR# | Tyr845 | autophosphorylated | 2231 |
| 23 | TK | EGFR | +control | unphosphorylated | 2245 |
| 24 | Cell cycle | P53# | Ser15 | ATR, DNA-PK, ATM | 9286 |
| 25 | Cell cycle | P53 | +control | unphosphorylated | 2524 |
| 26 | Cell cycle | Rb | Ser795 | cdks | 9301 |

#= a blocking peptide is available
Abbreviations used:
PKB, protein kinase B;
PTEN, phosphate and tensin homolog deleted on chromosome 10;
PTK, Protein Tyrosine Kinase;
MPAK, Mitogen Activated Protein Kinase;
MMAC, mutated in multiple advanced cancers;
TK, Tyrosine Kinase

TABLE 2
Some of the approximately 75 motif-specific antibodies that we propose to qualify for
phosphoprotein profiling in Frozen Tissue MicroArrays (‘+control’ binds the
unphosphorylated protein).

| | | | Kinase | | | |
|---|---|---|---|---|---|---|
| | ST pathway | | substrate | Qualified | Upstream | Supplier, |
| | profile | Phosphoprotein # | motif | applications | kinase(s) | Cat. No.* |
| 1 | Akt/survival | AKT1 | Ser308 | IHC | PDK1/2 | CST 9275 |
| 2 | Akt/survival | Rac1/cdc42 | Ser71 | WB | Akt | CST 2461 |
| 3 | Cell cycle | Chk1 | Ser345 | WB | DNA- | CST 2341 |
| | | | | | PK/ATM/ATR | |
| 4 | Cell cycle | Chk2 | The68 | WB | DNA- | CST 2661 |
| | | | | | PK/ATM/ATR | |
| 5 | Cell cycle | P53 | Ser20 | IHC | Chk1/2 | CST 9287 |
| 6 | Cell cycle | P53 | Ser392 | IHC | CAK | CST 9281 |
| 7 | Cell cycle | Cdc2 | Tyr15 | WB, IC | Myt1 | CST 9111 |
| 8 | Cell cycle | Cdc2 | +control | WB | Unphosphory. | CST 9112 |
| 9 | RAS-MAPK | c-Raf | Ser259 | IHC | Ras | CST 9421 |
| 10 | RAS-MAPK | c-myc | Thr58/Ser62 | IHC | MAPK, SAPK | CST 9401 |
| 11 | RAS-MAPK | SAPK/JNK | Thr183/Tyr185 | IHC | SEK1/MKK4 | CST 9255 |
| 12 | RAS-MAPK | SAPK/JNK | +control | WB, IC | Unphosphory. | CST 9252 |
| 13 | RAS-MAPK | ATF-2 | Thr71 | IHC | P38 MAPK | CST 9221 |
| 14 | RAS-MAPK | ATF-2 | +control | WB, IC | Unphosphory. | CST 9222 |
| 15 | RAS-MAPK | Elk-1 | Ser383 | IHC | P38 MAPK | CST 9181 |
| 16 | RAS-MAPK | SEK1/MKK4 | Thr261 | WB | MEKK1, MLKs, | CST 9151 |
| | | | | | ASK1 | |
| 17 | RAS-MAPK | c-Jun | Ser63 | IHC | SAPK/JNK | CST 9261 |
| 18 | RAS-MAPK | c-Jun | +control | WB | Unphosphory. | CST 9162 |
| 19 | RAS-MAPK | MKK3/MKK6 | Ser189/207 | WB | MLK3 | CST 9231 |
| 20 | RAS-MAPK | MKK3/MKK6 | +control | WB | Unphosphory. | CST 9232 |
| 21 | PKC | PKCα/βII | Thr638/641 | WB, IHC | Autophos. | CST 9375 |
| 22 | PKC | PKCδ | Thr505 | WB, IHC | PDK1 | CST 9374 |
| 23 | PKC | PKCμ | Ser744/748 | WB | PKC | CST 2054 |
| 24 | PKC | PDK/PKCμ | +control | WB | Unphosphory. | CST 2052 |
| 25 | PKC | PKCζ/λ | Thr410/403 | IHC | PDK1 | CST 9378 |

Additional abbreviations:
western blot, WB;

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An antibody protein analysis chip comprising:
a substrate comprising a plurality of layers wherein at least one layer is a conductive layer comprising a conductive material;
a main channel etched in the conductive layer which begins at a loading reservoir and ends at an outlet channel;
a plurality of side channels etched in the conductive layer at a substantially perpendicular angle to the main channel wherein the side channels are in communication with the main channel and end in at least one well; and
a protein-specific antibody confined in at least one well.

2. The analysis chip of claim 1 wherein the conductive material is gold.

3. The analysis chip of claim 1 wherein the conductive material is aluminum.

4. The analysis chip of claim 1 wherein the conductive material is chromium.

5. The analysis chip of claim 1 wherein the protein-specific antibody is engaged to a detectable tag.

6. The analysis chip of claim 5 wherein the detectable-tag is a quantum dot.

7. The analysis chip of claim 1 wherein the antibody is specific for a kinase peptide.

8. The analysis chip of claim 1 wherein the substrate is further engaged to a hydrophobic ink layer.

9. A antibody protein analysis chip comprising:
a substrate comprising a plurality of layers wherein at least one layer is a conductive layer comprising a conductive material;
an S-shaped channel etched in the conductive layer which begins at a loading reservoir and ends at an outlet channel;
a plurality of side channels etched in the conductive layer wherein the side channels are in communication with the main channel and end in at least one well;
a protein-specific antibody confined in at least one well; and
a battery in electrical communication with the substrate.

10. The analysis chip of claim 9 wherein the conductive material is gold.

11. The analysis chip of claim 9 wherein the conductive material is aluminum.

12. The analysis chip of claim 9 wherein the conductive material is chromium.

13. The analysis chip of claim 9 wherein the protein-specific antibody is engaged to a detectable tag.

14. The analysis chip of claim 13 wherein the detectable-tag is a quantum dot.

15. The analysis chip of claim 1 wherein the antibody is specific for a kinase peptide.

16. The analysis chip of claim 1 wherein the substrate is further engaged to a hydrophobic ink layer.

17. A kit comprising:
an antibody protein analysis chip comprising:
a substrate comprising a plurality of layers wherein at least one layer is a conductive layer comprising a conductive material;
an S-shaped channel etched in the conductive layer which begins at a loading reservoir and ends at an outlet channel;
a plurality of side channels etched in the conductive layer wherein the side channels are in communication with the main channel and end in a well;
an at least one protein-specific antibody confined in each well; and
a battery in electrical communication with the substrate; and
a cover engaged to the analysis chip wherein the cover comprises a plurality of openings corresponding to the wells.

18. The kit of claim 17 wherein the conductive material of the analysis chip is gold.

19. The kit of claim 17 wherein the antibodies in each well are engaged to a detectable tag.

20. The kit of claim 19 wherein the detectable tag is a quantum dot.
